(19) United States
(12) Patent Application Publication
(10) Pub. No.: US 2023/0098076 A1
(43) Pub. Date: Mar. 30, 2023

(54) A PROCESS FOR PREPARATION OF 3,6-DICHLOROCYANO PYRAZINE, 3,6-DIOXOPIPERAZINE DERIVATIVES AND PRODUCTION OF FAVIPIRAVIR THEREOF

(71) Applicant: COUNCIL OF SCIENTIFIC AND INDUSTRIAL RESEARCH (AN INDIAN REGISTERED BODY INCORPORATED UNDER THE REGN, Delhi (IN)

(72) Inventors: Raji Reddy CHADA, Hyderabad (IN); Amol DNYANDEV PATIL, Hyderabad (IN); Subbarao MUPPIDI, Hyderabad (IN); Nagender PUNNA, Hyderabad (IN); Ramachandra Reddy DONTHIRI, Hyderabad (IN); Ajay K SINGH, Hyderabad (IN); Prathama S MAINKAR, Hyderabad (IN); Chandrasekhar SRIVARI, Hyderabad (IN); Rajamannar THENNATI, Vadodara (IN)

(21) Appl. No.: 17/908,362

(22) PCT Filed: Jun. 11, 2021
(86) PCT No.: PCT/IN2021/050571

§ 371 Date: Aug. 31, 2022

(30) Foreign Application Priority Data
Jun. 12, 2020 (IN) 202011024682

(51) Int. Cl. C07D 241/24; C07D 241/08

(52) U.S. Cl. CPC C07D 241/24; C07D 241/08

(57) ABSTRACT

The present disclosure provides a process for preparation of 3,6-dichlorocyano pyrazine, 3,6-dioxopiperizine derivatives and production of favipiravir via ammonia or amine-mediated cyclization and chlorination using POCl3 in the presence of pyridine or PCl5. [Formula] wherein in 3,6-dioxopiperazine derivatives (III), X is CN, CONH2 or COOR2′, R1, R2 and R2′ are individually selected from H, C1-C12 alkyl, COOR3 and SO2R3 wherein R3 is substituted or unsubstituted linear or branched lower alkyl.

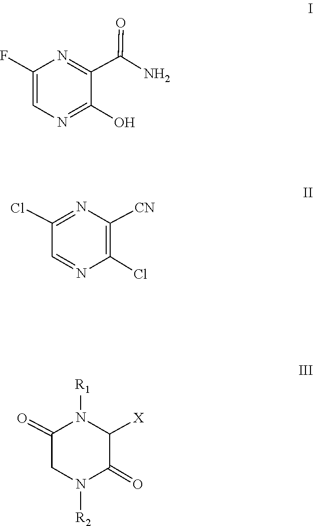

### FIELD OF THE INVENTION

The present invention relates to a process for preparation of 3,6-dichlorocyano pyrazine (II), 3,6-dioxopiperazine derivatives (III) and production of favipiravir (I), in particular, to a process for the preparation of 3,6-dichlorocyano pyrazine using POCl₃ in the presence of pyridine or PCl₅ from 3,6-dioxopiperazine derivatives, which in turn prepared via ammonia-mediated cyclization as key steps, leading to the production of favipiravir.

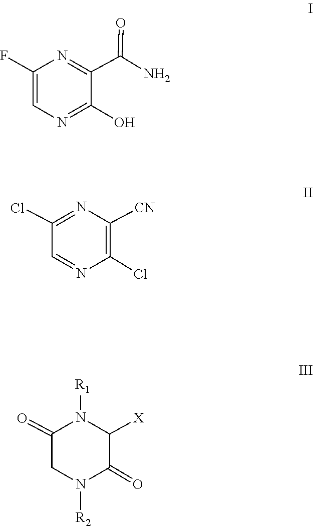

wherein in Formula III, X is CN, CONH₂ or COOR₂′, R₁, R₂ and R₂′ are individually selected from H, C1-C12 alkyl, COORS and SO₂R₃ wherein R₃ is substituted or unsubstituted linear or branched lower alkyl.

### BACKGROUND OF THE INVENTION

In early 2020, World Health Organization had declared that a novel type of SARS-Cov-2 virus named COVID-19 having pneumonia kind of symptoms is a global pandemic, which had emerged in the city of Wuhan, China and is spreading rapidly across the world. To the date (May 5, 2020) worldwide, 35,17,345 COVID-19 infected patients and 2,43,401 deaths are confirmed. These huge numbers aroused with in the period of 5 months, which denotes that the present novel corona virus is awfully dangerous. Unfortunately, up to now there is no treatment available for this COVID-19 virus. However, some of the observational studies of COVID-19 patients have been reported that the anti-viral drugs approved by the FDA for Ebola, malaria and influenza are effectively working in the outcome of novel corona virus patients. At present, remedesivir and favipiravir are in the top place among all the anti-viral drugs, which could shorten the time to recovery of COVID-19 infection.

In this context, favipiravir (T-705; 6-fluoro-3-hydroxy-2-pyrazinecarboxamide) is an antiviral drug that selectively inhibits the RNA-dependent RNA polymerase (RdRp) of RNA viruses. It was developed by Toyama Chemical Co. Ltd and approved in Japan with the brand name of Avigan in 2014. Animal studies revealed that the favipiravir is also active to treat different kind of other viruses such as yellow fever, West Nile virus and Ebola. At present, phase-III clinical trials of favipiravir for the treatment Covid-19 are going on.

To date, several methods has been reported for the synthesis of favipiravir, among that synthesis of favipiravir via 3,6-dichloropyrazine-2-carbonitrile is the advanced intermediate and industrially more favorable (PCT 2010087117, CN 106588786, CN 106478528, Chemical Papers, 73(5), 1043-1051; 2019). Mainly two routes are available for the preparation of 3,6-dichloropyrazine-2-carbonitrile via the chlorination of 6-bromo-3-hydroxypyrazine-2-carboxamide as a common intermediate. Synthetic scheme 1 uses the 3-aminopyrazine-2-carboxylic acid as a key starting material to the preparation of common intermediate 6-bromo-3-hydroxypyrazine-2-carboxamide by esterification, bromination, diazotization and amidation.

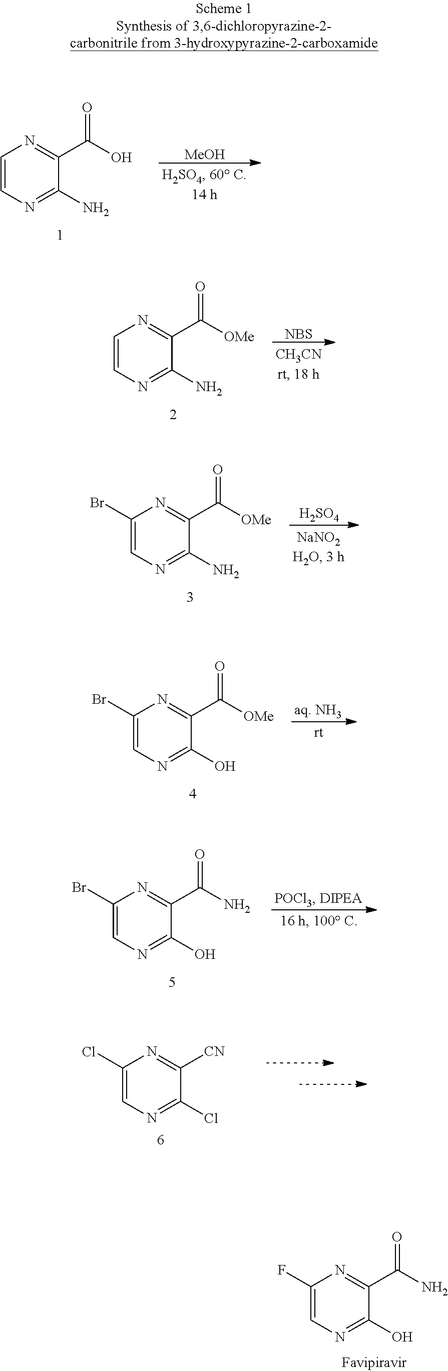

Synthetic scheme 2, uses the dimethyl 2-aminomalonate as a starting material to prepare the 6-bromo-3-hydroxypyrazine-2-carboxamide by amidation, condensation and bromination. However, the unsafe reagents and lower yields used in the above routes are industrially not suitable for producing commercially viable product of favipiravir.

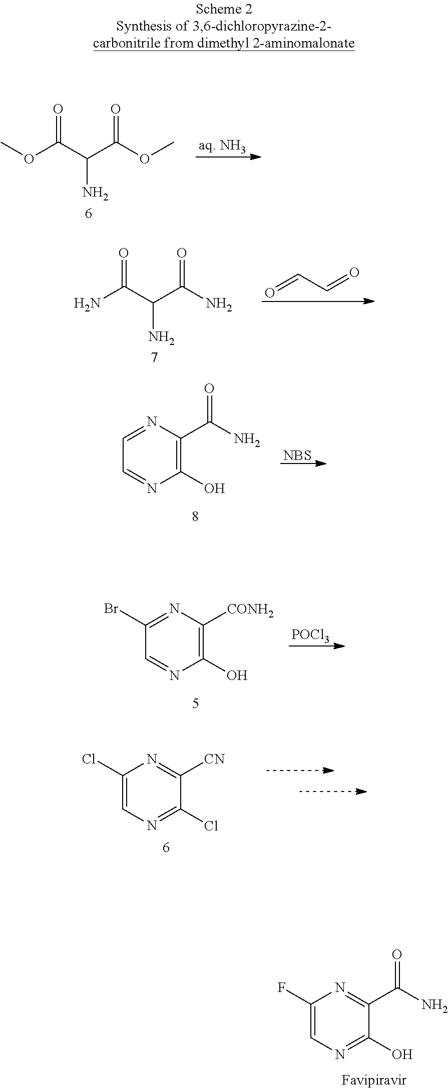

Moreover, availability and preparation of key starting material 3-hydroxypyrazine carboxamide 1 requires a number of steps and also tedious, which adds to the price of final product favipiravir. Therefore, improvement in the yield for 3,6-dichloropyrazine-2-carbonitrile by overcoming the above problems in lesser number of steps is important for the production of favipiravir to make at commercially viable cost with low burden to environment.

In view of the limitations in the known art, there is requirement of a cost-effective, with atom-economy and scalable process for the production of highly pure 3,6-dichlorocyano pyrazine, 3,6-dioxopiperazine derivatives, which serve as key intermediates leading to the production of favipiravir.

### Objective of the Invention

The main objective of the present invention is to provide a cost-effective, with atom-economy and scalable process for the production of highly pure 3,6-dichlorocyano pyrazine, 3,6-dioxopiperazine derivatives as mentioned above, which serve as key intermediates leading to the production of favipiravir.

Another objective of the present invention is to provide a process for obtaining the key intermediates 3,6-dichlorocyano pyrazine and 3,6-dioxopiperazine derivatives as mentioned above, by simple reaction protocol employing ammonia and POCl₃ in the presence of pyridine or PCl₅, respectively as reagents.

Yet another objective of the present invention is to provide an effective process for the production of favipiravir via formation of highly pure 3,6-dioxopiperazine 2-carboxamide/carbonitrile and 3,6-dichlorocyano pyrazine as intermediates in the process protocol.

### SUMMARY OF THE INVENTION

In an aspect of the present disclosure, there is provided a process for preparation of 3,6-dichlorocyano pyrazine of formula II,

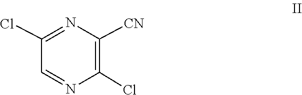

comprising the steps of:
(a) chlorination of 3,6-dioxopiperazine derivative of formula III with POCl₃ and pyridine or PCl₅ at a temperature in the range of 90-140° C. for 4-20 hours,

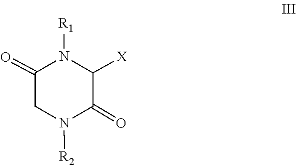

wherein X is CN, CONH₂ or COOR₂′, R₁, R₂ and R₂′ are individually selected from H, C1-C12 alkyl, COOR₃ and SO₂R₃ wherein R₃ is substituted or unsubstituted linear or branched lower alkyl, to obtain a compound of Formula II and
(b) purification of the compound of Formula II obtained in step (a).

In another aspect of the present disclosure, there is provided a process for preparation of 3,6-dioxoninerazine derivatives of formula III,

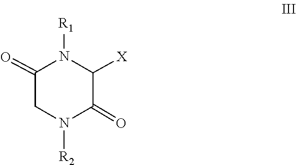

wherein, X is CN, CONH₂ or COOR₂′, R₁, R₂ and R₂′ are individually selected from H, C1-C12 alkyl, COOR₃ and SO₂R₃ wherein R₃ is substituted or unsubstituted linear or branched lower alkyl, comprising the steps of:
(a) cyclization of halo-amide of formula V

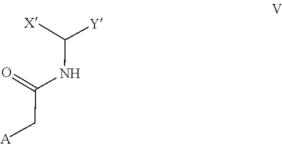

wherein X′ and Y′ are individually selected from CN, CONH₂ and COOR₃′, where R₃′ is selected from H and C1-C12 alkyl, A is selected from Cl, Br, OH and OR₅ wherein R₅ is SO₂R₄ and R₄ is substituted or unsubstituted linear or branched lower alkyl, with alcoholic ammonia or amine derivative at a temperature in the range of 60-100° C. for 10-24 hours to obtain a compound of Formula III, and
(b) filtration and recrystallization of the compound of Formula III obtained in step (a).

In one another aspect of the present disclosure, there is provided a process for preparation of compound of formula I

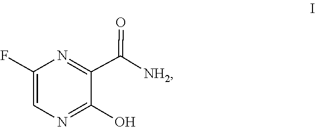 [SMILES: NC(=O)c1nc(F)cnc1O]

which comprises the steps of:
(a) cyclization of halo-amide of formula V

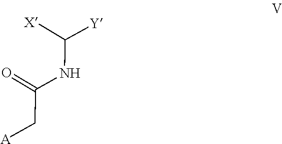

wherein X′ and Y′ are individually selected from CN, CONH₂ and COOR₃′, where R₃′ is selected from H and C1-C12 alkyl, A is selected from Cl, Br, OH and OR₅ wherein R₅ is SO₂R₄ and R₄ is substituted or unsubstituted linear or branched lower alkyl,
with alcoholic ammonia or amine derivatives at a temperature in the range of 60-100° C. for 10-24 hours to obtain a compound of Formula III,

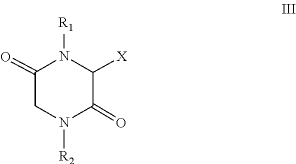

wherein, X is CN, CONH₂ or COOR₂′, R₁, R₂ and R₂′ are individually selected from H, C1-C12 alkyl, COORS and SO₂R₃ wherein R₃ is substituted or unsubstituted linear or branched lower alkyl,
(b) chlorination of 3,6-dioxopiperazine derivative of formula III with POCl₃ and pyridine or PCl₅ at a temperature in the range of 90-140° C. for 4-20 hours, to obtain a compound of Formula II;

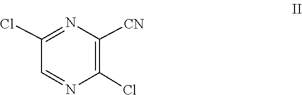

(c) fluorination of compound of formula II obtained in step (b) with potassium fluoride and PTC in a solvent to obtain difluorocyano pyrazine of formula VII at a temperature in the range of 50° C. to 70° C.;

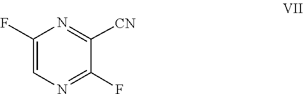

(d) functionalization of aromatic ring in the compound of formula VII obtained in step (c) from fluorine to hydroxy in the presence of sodium acetate to obtain 6-fluoro-3-hydroxypyrazine-2-carbonitrile of formula VIII;

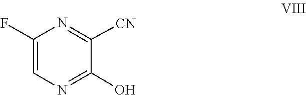

and
(e) hydrolysis of cyano functionality of formula VIII to amide in presence of H₂O₂ and NaOH solution to obtain compound of Formula (I).

These and other features, aspects, and advantages of the present subject matter will be better understood with reference to the following description and appended claims. This summary is provided to introduce a selection of concepts in a simplified form. This summary is not intended to identify key features or essential features of the claimed subject matter, nor is it intended to be used to limit the scope of the claimed subject matter.

### DETAILED DESCRIPTION OF THE INVENTION

The invention will now be described in detail in connection with certain preferred and optional embodiments, so that various aspects thereof may be more fully understood and appreciated.

### Definitions

For convenience, before further description of the present disclosure, certain terms employed in the specification, and examples are delineated here. These definitions should be read in the light of the remainder of the disclosure and understood as by a person of skill in the art. The terms used herein have the meanings recognized and known to those of skill in the art, however, for convenience and completeness, particular terms and their meanings are set forth below.

The articles “a”, “an” and “the” are used to refer to one or to more than one (i.e., to at least one) of the grammatical object of the article.

The terms “comprise” and “comprising” are used in the inclusive, open sense, meaning that additional elements may be included. It is not intended to be construed as “consists of only”.

Throughout this specification, unless the context requires otherwise the word “comprise”, and variations such as “comprises” and “comprising”, will be understood to imply the inclusion of a stated element or step or group of element or steps but not the exclusion of any other element or step or group of element or steps.

Ratios, concentrations, amounts, and other numerical data may be presented herein in a range format. It is to be understood that such range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited. For example, a temperature in the range of 90° C. to 140° C. should be interpreted to include not only the explicitly recited limits of 90° C. to 140° C. but also to include sub-ranges, such as 95° C. to 106° C., and so forth, as well as individual amounts, within the specified ranges, such as 112.7° C., and 135.5° C.

As discussed in the background, there are drawbacks associated with the existing process of synthesis of favipiravir, in terms of poor yield of final product, use of unsafe reagents, large number of tedious reaction steps, availability of key starting materials, commercial and industrial unsuitability of the non-scalable process and high cost. Favipiravir, among all the anti-viral drugs, is useful in shortening the recovery time for people infected with COVID-19. Thus, an easy, low cost and scalable preparation process of favipiravir is an essential need. The present disclosure provides a cost-effective, with atom-economy and scalable process for the production of highly pure favipiravir, and 3,6-dichlorocyano pyrazine, 3,6-dioxopiperazine derivatives, which serve as key intermediates leading to the production of favipiravir. The process employs easy reaction parameters that can be scalable to large scale production of favipiravir and its intermediates of formula II and formula III.

The present disclosure is not to be limited in scope by the specific embodiments described herein, which are intended for the purposes of exemplification only. Functionally-equivalent products, compositions, and methods are clearly within the scope of the disclosure, as described herein.

In an embodiment of the present disclosure, there is provided a process for the preparation of 3,6-dichlorocyano pyrazine of formula II,

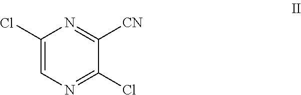

comprising the steps of:
(a) chlorination of 3,6-dioxopiperazine derivative of formula III with POCl₃ and pyridine or PCl₅ at a temperature in the range of 90-140° C. for 4-20 hours,

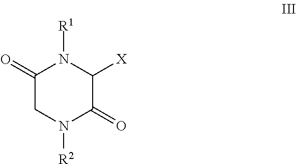

wherein X is CN, CONH₂ or COOR₂′, R₁, R₂ and R₂′ are individually selected from H, C1-C12 alkyl, COORS and SO₂R₃ wherein R₃ is substituted or unsubstituted linear or branched lower alkyl, to obtain a compound of Formula II and
(b) purification of the compound of Formula II obtained in step (a).

In an embodiment of the present disclosure, there is provided a process for preparation of 3,6-dichlorocyano pyrazine of formula II, wherein the purification method is selected from crystallization, filtration, and chromatography.

In an embodiment of the present disclosure, there is provided a process for the preparation of 3,6-dichlorocyano pyrazine of formula II,

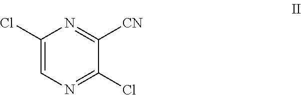

comprising the steps of:
(a) chlorination of 3,6-dioxopiperazine derivative of formula III with POCl₃ and pyridine or PCl₅ at a temperature in the range of 90-140° C. for 4-20 hours,

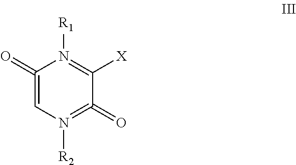

wherein X is CN, CONH₂ or COOR₂′, R₁, R₂ and R₂′ are individually selected from H, C1-C12 alkyl, COOR₃ and SO₂R₃ wherein R₃ is substituted or unsubstituted linear or branched lower alkyl, to obtain a compound of Formula II and
(b) purification of the compound of Formula II obtained in step (a), wherein the purification method is selected from crystallization, filtration, and chromatography.

In an embodiment of the present disclosure, there is provided a process for preparation of 3,6-dioxopiperazine derivatives of formula III

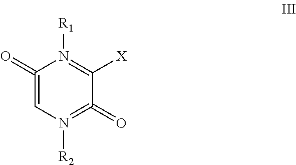

wherein, X is CN, CONH₂ or COOR₂′, R₁, R₂ and R₂′ are individually selected from H, C1-C12 alkyl, COOR₃ and SO₂R₃ wherein R₃ is substituted or unsubstituted linear or branched lower alkyl, comprising the steps of:
(a) cyclization of halo-amide of formula V

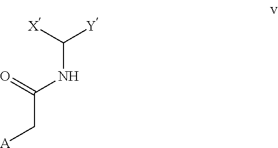

wherein X′ and Y′ are individually selected from CN, CONH₂ and COOR₃′, where R₃′ is selected from H and C1-C12 alkyl, A is selected from Cl, Br, OH and OR₅ wherein R₅ is SO₂R₄ and R₄ is substituted or unsubstituted linear or branched lower alkyl,
with alcoholic ammonia or amine derivative at a temperature in the range of 60-100° C. for 10-24 hours to obtain a compound of Formula III, and
(b) filtration and recrystallization of the compound of Formula III obtained in step (a).

In an embodiment of the present disclosure, there is provided a process for preparation of 3,6-dioxopiperazine derivatives of formula III, wherein the alcoholic ammonia is methanolic ammonia, or ethanolic ammonia; and the amine derivative is selected from alkyl, cycloalkyl, or benzyl amines, carbamates, and sulphonamides. In another embodiment of the present disclosure, the alcoholic ammonia is methanolic ammonia.

In an embodiment of the present disclosure, there is provided a process for preparation of 3,6-dioxopiperazine derivatives of formula III, wherein the solvent system for recrystallization is selected from alcohol as a single solvent, or a two solvent mixtures, comprising a water:alcohol system. In another embodiment of the present disclosure, the solvent system for recrystallization is a two solvent mixtures, comprising a water:alcohol system.

In an embodiment of the present disclosure, there is provided a process for preparation of 3,6-dioxopiperazine derivatives of formula III, wherein the halo-amide of formula V,

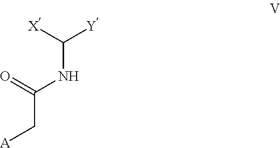

wherein X′ and Y′ are individually selected from CN, CONH₂ and COOR₃′, where R₃′ is selected from H and C1-C12 alkyl, A is selected from Cl, Br, OH and OR₅ wherein R₅ is SO₂R₄ and R₄ is substituted or unsubstituted linear or branched lower alkyl, is prepared by acylation reaction between the compound of formula IV and chloroacetyl chloride of formula VI in presence of base at room temperature,

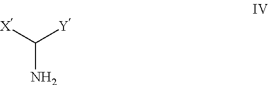

wherein X′ and Y′ are as defined above;

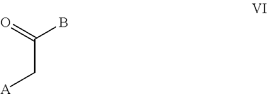

wherein A is as defined above and B is selected from Cl, Br, OH and OR₅ wherein R₅ is SO₂R₄ and R₄ is substituted or unsubstituted linear or branched lower alkyl.

In an embodiment of the present disclosure, there is provided a process for preparation of 3,6-dichlorocyano pyrazine of formula II

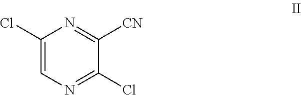

which comprises the steps of:
(a) acylation reaction between the compound of formula IV and chloroacetyl chloride of formula VI in presence of base at room temperature to obtain halo-amide of formula V,

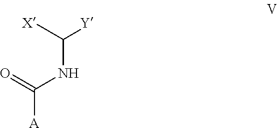

wherein X′ and Y′ are individually selected from CN, CONH₂ and COOR₃′, where R₃′ is selected from H and C1-C12 alkyl, A is selected from Cl, Br, OH and OR₅ wherein R₅ is SO₂R₄ and R₄ is substituted or unsubstituted linear or branched lower alkyl,

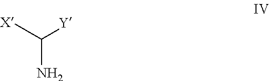

wherein X′ and Y′ are as defined above;

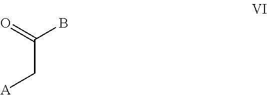

wherein A is as defined above and B is selected from Cl, Br, OH and OR₅ wherein R₅ is SO₂R₄ and R₄ is substituted or unsubstituted linear or branched lower alkyl;
(b) cyclization of halo-amide of formula V with alcoholic ammonia or amine derivative, at a temperature in the range of 60-100° C. for 10-24 hours to obtain a compound of Formula III,

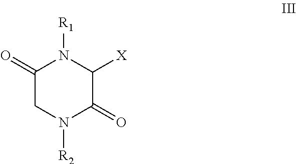

wherein, X is CN, CONH₂ or COOR₂′, R₁, R₂ and R₂′ are individually selected from H, C1-C12 alkyl, COORS and SO₂R₃ wherein R₃ is substituted or unsubstituted linear or branched lower alkyl,
(c) chlorination of 3,6-dioxopiperazine derivative of formula III with POCl₃ and pyridine or PCl₅ at a temperature in the range of 90-140° C. for 4-20 hours, to obtain a compound of Formula II and
(d) purification of the compound of Formula II.

In an embodiment of the present disclosure, there is provided a process for preparation of 3,6-dichlorocyano pyrazine of formula II, wherein the alcoholic ammonia is methanolic ammonia, or ethanolic ammonia; and the amine derivative is selected from alkyl, cycloalkyl, or benzyl amines, carbamates, and sulphonamides.

In an embodiment of the present disclosure, there is provided a process for preparation of 3,6-dichlorocyano pyrazine of formula II, wherein the purification method is selected from crystallization, filtration and chromatography.

In an embodiment of the present disclosure, there is provided a process for preparation of compound of formula I

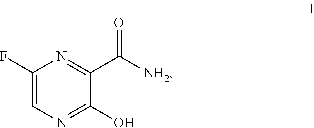 [SMILES: NC(=O)c1nc(F)cnc1O]

which comprises the steps of:
(a) cyclization of halo-amide of formula V

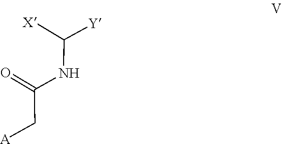

wherein X′ and Y′ are individually selected from CN, CONH₂ and COOR₃′, where R₃′ is selected from H and C1-C12 alkyl, A is selected from Cl, Br, OH and OR₅ wherein R₅ is SO₂R₄ and R₄ is substituted or unsubstituted linear or branched lower alkyl,
with alcoholic ammonia or amine derivatives at a temperature in the range of 60-100° C. for 10-24 hours to obtain a compound of Formula III,

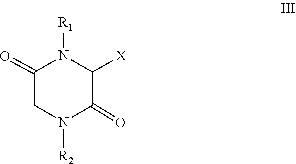

wherein, X is CN, CONH₂ or COOR₂′, R₁, R₂ and R₂′ are individually selected from H, C1-C12 alkyl, COOR₃ and SO₂R₃ wherein R₃ is substituted or unsubstituted linear or branched lower alkyl,
(b) chlorination of 3,6-dioxopiperazine derivative of formula III with POCl₃ and pyridine or PCl₅ at a temperature in the range of 90-140° C. for 4-20 hours, to obtain a compound of Formula II;

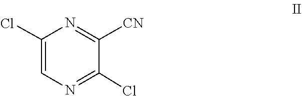

(c) fluorination of compound of formula II obtained in step (b) with potassium fluoride and PTC in a solvent to obtain difluorocyano pyrazine of formula VII at a temperature in the range of 50° C. to 70° C.;

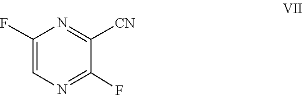

(d) functionalization of aromatic ring in the compound of formula VII obtained in step (c) from fluorine to hydroxy in the presence of sodium acetate to obtain 6-fluoro-3-hydroxypyrazine-2-carbonitrile of formula VIII;

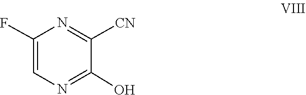

and
(e) hydrolysis of cyano functionality of formula VIII to amide in presence of H₂O₂ and NaOH solution to obtain compound of Formula (I).

In an embodiment of the present disclosure, there is provided a process for preparation of compound of formula I, wherein the alcoholic ammonia is methanolic ammonia, or ethanolic ammonia; and the amine derivative is selected from alkyl, cycloalkyl, or benzyl amines, carbamates, and sulphonamides.

In an embodiment of the present disclosure, there is provided a process for preparation of compound of formula I, wherein the solvent used in step (c) is selected from DMF, and DMSO. In another embodiment of the present disclosure, the solvent used in step (c) is DMF.

### EXAMPLES

The present disclosure provides a process for the synthesis of easily scalable 3,6-dichloropyrazine-2-carbonitrile and 3,6-dioxopiperazine derivatives, in particular 3,6-dioxopiperazine-2-carboxamide intermediates, Favipiravir and analogs thereof, comprising the steps as defined in the detailed description. The synthesis of representative compounds has been given.

Scheme 3 represents the process steps for the preparation of 3,6-dichlorocyano pyrazine (Formula II), 3,6-dioxopiperazine derivative (Formula III), in particular 3,6-dioxopiperazine-2-carboxamide, Favipiravir and their analogs.

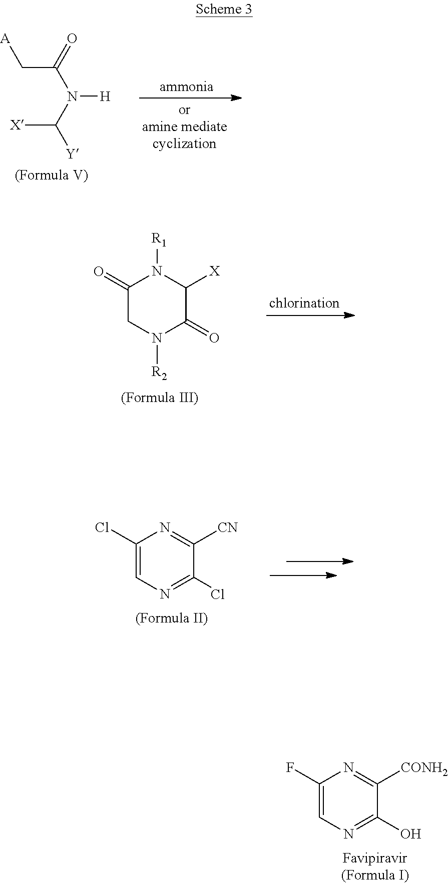

Wherein in the above scheme X is CN, CONH₂ or COOR₂′, R₁, R₂ and R₂′ are individually selected from H, C1-C12 alkyl, COOR₃ and SO₂R₃ wherein R₃ is substituted or unsubstituted linear or branched lower alkyl, X′ and Y′ are individually selected from CN, CONH₂ and COOR₃′, where R₃′ is selected from H and C1-C12 alkyl, A is selected from Cl, Br, OH and OR₅ wherein R₅ is SO₂R₄ and R₄ is substituted or unsubstituted linear or branched lower alkyl.

The process with specific reactants and intermediates could be represented in Scheme 4 as follows:

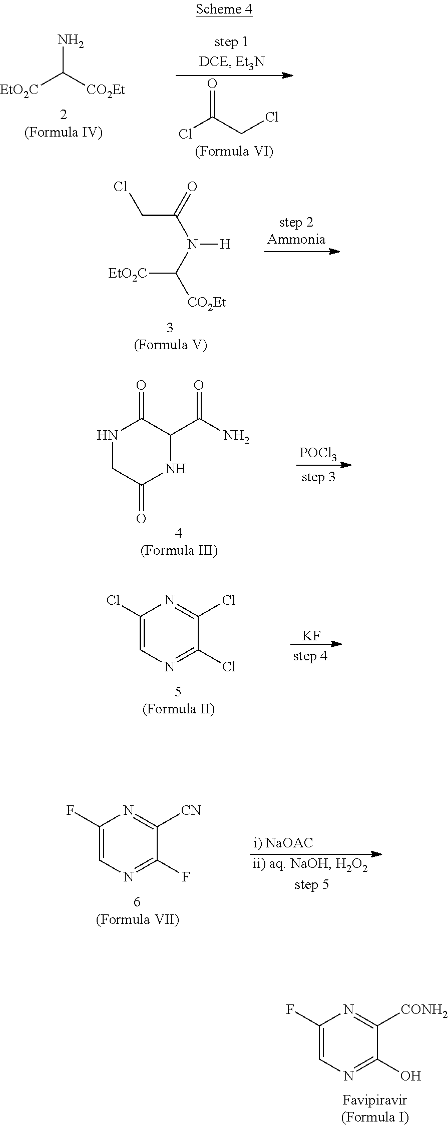

The process preparation of 3,6-dichlorocyano pyrazine (Formula II), 3,6-dioxopiperazine derivatives (Formula III), and production of favipiravir via ammonia or amine-mediated cyclization and chlorination using POCl₃ in the presence of pyridine or PCl₅ as key steps as illustrated in scheme 4 is described as follows. This process is the most effective and convenient method to produce in high yields, purity and would be economical at industrial scale.

This newly established process as mentioned in scheme 4 starts from compound of formula IV involving a two-step reaction sequence and comprises of the following simple and easy to replicate in large scale operations: acylation, ammonia-mediated cyclization as shown in scheme 4 to give the desired compounds of formula III.

The process route of the present disclosure can be completed very efficiently in five total steps with a short reaction time and a highly feasible strategy which could be most suitable for the industrial scale production of Favipiravir. Further, this process is also suitable for the generation of a large library of intermediates which may also find interesting properties.

The first step of this route contains acylation, wherein diverse functionalization is possible with the use of various substrates. While, these amides could serve as valued intermediates, to produce yet another library of 3,6-dioxopiperizine derivatives upon treatment with ammonia or amine derivatives. Further, the halogenation could be accomplished by variation of halogenation reagents to provide the subsequent 3,6-dihalopyrazine derivatives in excellent yields. Then, halogen exchange with fluorine using fluorinating agent could be performed in the presence of phase-transfer agent to generate 3,6-difluoropyrazine-2-carbonitrile, which could be converted in Favipiravir through conversion of 3-fluoro group to hydroxyl and cyano hydrolysis to amide under hydrolysis conditions. All the reaction steps include purification and methodical characterization of the single reaction product at every stage of the process, making it very much viable for production scale.

The initial step of the present invention is acylation reaction between the compound of formula IV,

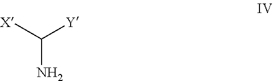

wherein X′ and Y′ are individually selected from CN, CONH₂ and COOR₃′, where R₃′ is selected from H and C1-C12 alkyl;
and chloroacetyl chloride compound of formula VI

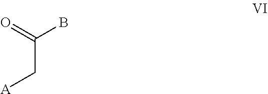

wherein A is selected from Cl, Br, OH and OR₅ wherein R₅ is SO₂R₄ and R₄ is substituted or unsubstituted linear or branched lower alkyl and B is selected from Cl, Br, OH and OR₅ wherein R₅ is SO₂R₄ and R₄ is substituted or unsubstituted linear or branched lower alkyl; in presence of base at room temperature to furnish the compounds represented by formula V

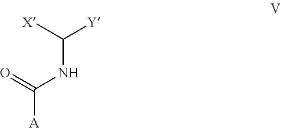

wherein X′, Y′ and A are as defined above.

The second step in the process is cyclization reaction of formula V obtained in the step (i) with ammonia (NH₃) or amine derivatives to afford the 3,6-dioxopiperazine derivative formula III.

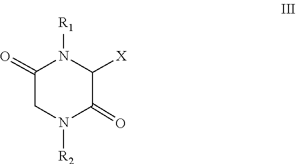

wherein, X is CN, CONH₂ or COOR₂′, R₁, R₂ and R₂′ are individually selected from H, C1-C12 alkyl, COOR₃ and SO₂R₃ wherein R₃ is substituted or unsubstituted linear or branched lower alkyl and wherein the amine derivative is selected from alkyl or cycloalkyl amines, carbamates and sulphonamides. In this embodiment, the temperature ranges from 60° C. to 120° C., preferably at 100° C. for the cyclization and about five volumes of the alcoholic ammonia. Wherein the alcoholic ammonia is methanolic ammonia or ethanolic ammonia.

The third step of the process is, chlorination reaction of formula III obtained in step (ii) with phosphorous oxychloride and pyridine or PCl₅ at 90-140° C. to furnish the dichlorocyano pyrazine of formula II.

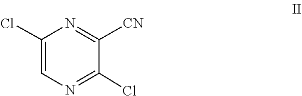

The fourth step of the process is, fluorination reaction of formula II obtained in step (iii) with potassium fluoride and PTC Tetrabutyl ammonium bromide or crown ether to deliver the difluorocyano pyrazine formula VII. In this embodiment different solvents such as DMF and DMSO are screened, wherein DMF affords higher yield. The temperature requiring of about 50° C. to 70° C. for the reaction.

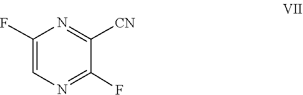

The final step of the present invention is the preparation of Favipiravir (formula I), from formula VII afforded in step (iv), from fluorine to hydroxy in the presence of sodium acetate at about 60° C. followed by hydrolysis of cyano functionality to amide in presence of 30% H₂O₂ and 6% NaOH solution.

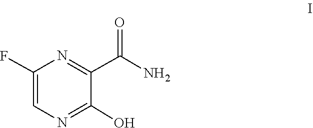 [SMILES: NC(=O)c1nc(F)cnc1O]

The embodiments of the present invention will be more specifically explained by following examples. However, the following examples are given by way of illustration and the scope of the present invention is not limited to the scope of these examples.

### Example 1: Preparation of Compound of 3 (Formula V)

80 g of diethyl 2-aminomalonate (2, prepared from diethyl malonate) was suspended in 1.2 mL of dichloroethane and was gradually added, 56.8 g of chloroacetylchloride and 190 mL of triethylamine and stirred at room temperature. Then, the reaction mixture was diluted with 800 mL of water; organic layer was separated and washed by 400 mL of saturated sodium bicarbonate solution. The organic layer was dried on rotary evaporator to afford the chloroacetyl diester (3, Formula V) in 104 g as white solid, >97% purity). This process step can be carried out using other reagents such as bromoacetyl bromide haloacetic acid or tosyl/mesyloxy acetyl halide or tosyl/mesyloxy acetic acid.

Mol. Formula: C₉H₁₄ClNO₅; Mp: 95-97° C.; ¹H NMR (400 MHz, CDCl₃) δ 7.51 (d, J=5.2 Hz, 1H), 5.14 (d, J=6.9 Hz, 1H), 4.33-4.24 (m, 4H), 4.11 (s, 2H), 1.32 (t, J=7.1 Hz, 6H); ¹³C NMR (101 MHz, CDCl₃) δ 165.9, 165.7, 62.9, 56.6, 42.1, 14.01; HRMS: calcd. for C₉H₁₄ClNO₅ [M+Na]⁺ 274.0458, found 274.0464.

### Example 2: Preparation of Compound of 4 (Formula III)

To 25 g of chloroacetyldiester (3, Formula V), 125 mL methanolic ammonia (7 N) was added and the solution was stirred between 60-120° C. and the stirring continued till the completion of the starting material. The reaction mixture was filtered and obtained crude product was recrystallized to afford 11 g of the 3,6-dioxopiperazine-2-carboxamide (4, Formula III) as off-white solid. This step can be carried out using other amines such as ammonia or allyl/benzyl amine and sulphonamide.

Mol. Formula: C₅H₇N₃O₃ mp: 260-262° C.; ¹H NMR (400 MHz, DMSO) δ 8.23 (d, J=2.8 Hz, 1H), 8.14 (d, J=2.1 Hz, 1H), 7.72 (s, 1H), 7.38 (s, 1H), 4.26 (d, J=3.2 Hz, 1H), 3.87 (d, J=17.2 Hz, 1H), 3.56 (dd, J=17.2, 3.5 Hz, 1H); ¹³C NMR (101 MHz, DMSO) δ 169.2, 167.3, 164.4, 59.7, 44.9; HRMS: calcd. for C₅H₈N₃O₃[M+H]⁺ 158.0566, found 158.0568.

### Example 3: Preparation of Compound of 5 (Formula II)

To the stirred solution of 10 g of 3,6-dioxopiperazine-2-carboxamide (4) in 100 mL of POCl₃ and 50 mL of pyridine stirred at 120° C. until completion of the starting material. The reaction mixture was poured into crushed ice, and extracted with 300 mL of ether. Combined organic layer were washed by 100 mL of saturated brine. The organic layer was dried over sodium sulphate, concentrated on rotary evaporator and obtained solid was purified by column chromatography using 100-200 mesh silica gel to afford the 3,6-dichlorocyano pyrazine (5) in 65% yield (7.2 g) as a white to pale-yellow solid. This process step has also been carried out using PCl₅ to obtain the desired product.

Mol. Formula: C₅HCl₂N₃: mp: 90-92° C.; ¹H NMR (400 MHz, DMSO-d) δ 9.03 (s, 1H); ¹³C NMR (101 MHz, DMSO) δ 149.63, 148.69, 146.97, 128.79, 114.10.

### Example 4: Preparation of Compound of 6 (Formula VII)

18.1 g of pre-dried potassium fluoride was placed in flask, followed by addition of 6.7 g of the TBAB (phase transfer catalyst) and 9 g of 3,6-dichloro-2-cyanopyrazine 5. Then, 54 mL of dry DMF or DMSO was added to the reaction mixture and stirred for 3 hours. After completion of starting material, reaction mixture was quenched with water, then extracted with 100 mL of ether and concentrated on rotary evaporator. The obtained reaction mixture was filtered through silica gel to afford the 3,6-difluoro-2-cyanopyrazine 6 in 87% yield (6.4 g) as a white solid.

Mol. Formula: C₅HF₂N₃; ¹H NMR (400 MHz, CDCl₃) δ 8.34 (dd, J=8.1, 1.5 Hz, 1H); ¹³C NMR (101 MHz, CDCl₃) δ 158.96 (d, J=210.6 Hz), 156.40 (d, J=210.1 Hz), 135.12 (dd, J=41.7, 11.1 Hz), 114.00 (dd, J=35.8, 11.3 Hz), 110.64 (d, J=8.9 Hz); ¹⁹F NMR (376 MHz, CDCl₃) δ −77.22 (d, J=37.1 Hz, 1F), −81.18 (d, J=37.1 Hz, 1F).

### Example 5: Preparation of Compound of 7 (Formula I)

6 g of 3,6-difluoropyrazine-2-carbonitrile 6 was dissolved in 60 mL of dioxane/water in 1:1 ratio, then 7 g of NaOAc was added to the reaction mixture and stirred at 60° C. After completion of starting material, the reaction mixture was concentrated and diluted with water. Afterward, aqueous layer was acidified with 2N HCl up to pH=2-3, extracted twice with 100 mL of ethyl acetate. The combined organic layers were concentrated on rotary evaporator to afford the 6-fluoro-3-hydroxypyrazine-2-carbonitrile as solid. To 4.5 g of this compound in 23 mL of 6.5% NaOH aqueous solution was added 3 mL of 30% H₂O₂ solution drop wise. After completion of starting material, the reaction mixture was acidified with HCl up to pH=2-3. The formed solid was filtered and washed with 2N HCl and dried to get the desired compound Favipiravir in 85% yield (4.26 g) as a pale yellow solid, which was further recrystallized in ethanol to get the >99% pure compound.

Mol. Formula: C₅H₄FN₃O₂; Mp: 186-188° C.; ¹H-NMR (400 MHz): δ 13.40 (s, 1H), 8.73 (s, 1H), 8.50 (d, J=7.97 Hz, 2H); ¹³C-NMR (101, MHz): δ 169.19, 160.21, 152.90 (d, J=243.4 Hz), 136.27 (d, J=43.3 Hz), 122.84. HRMS: calcd. for C₅H₄FN₃O₂ [M+H]⁺ 158.0366, found 158.0368.

### Advantages of the Present Disclosure

In view of the importance and limitations of efficient scalable production methods for preparation of 3,6-dichlorocyano pyrazine, 3,6-dioxopiperazine derivatives and production of favipiravir, the process of the present disclosure provides a highly effective and scalable manufacture method for the synthesis of 3,6-dichlorocyano pyrazine, 3,6-dioxopiperazine derivatives, and production of favipiravir.

The various advantages of the present process are given below.

The present disclosure provides an efficient process for the preparation of 3,6-dichlorocyano pyrazine, 3,6-dioxopiperazine derivatives and production of favipiravir.

Another advantage of the present disclosure is that the process could be operated via ammonia or amine-mediated cyclization and chlorination using and POCl₃ in the presence of pyridine or PCl₅ as key step leading to formation of 3,6-dioxopiperazine derivatives and dichlorocyano pyrazine, respectively as intermediates.

Further the present disclosure employs simpler reaction parameters amenable for large scale to achieve the production of Favipiravir, 3,6-dichlorocyano pyrazine of Formula II and 3,6-dioxopiperazine derivatives of Formula III.

Isolation and/or purification of the products obtained in the process of the present disclosure are easy and straightforward.

The present disclosure provides an attractive, with atom-economy, cost-effective and scalable method for the production of favipiravir.

We claim:

1. A process for preparation of 3,6-dichlorocyano pyrazine of formula II, 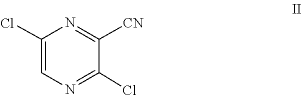
comprising the steps of:
(a) chlorination of 3,6-dioxopiperazine derivative of formula III with POCl₃ and pyridine or PCl₅ at a temperature in the range of 90-140° C. for 4-20 hours, 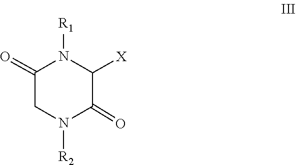
wherein X is CN, CONH₂ or COOR₂′, R₁, R₂ and R₂′ are individually selected from H, C1-C12 alkyl, COOR₃ and SO₂R₃ wherein R₃ is substituted or unsubstituted linear or branched lower alkyl, to obtain a compound of Formula II and
(b) purification of the compound of Formula II obtained in step (a).

2. The process as claimed in claim 1, wherein the purification method is selected from crystallization, filtration, and chromatography.

3. A process for preparation of 3,6-dioxopiperazine derivatives of formula III, 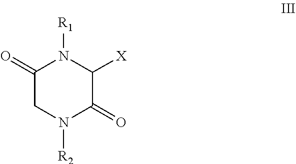
wherein, X is CN, CONH₂ or COOR₂′, R₁, R₂ and R₂′ are individually selected from H, C1-C12 alkyl, COOR₃ and SO₂R₃ wherein R₃ is substituted or unsubstituted linear or branched lower alkyl, comprising the steps of:
(a) cyclization of halo-amide of formula V 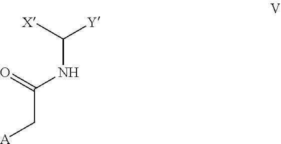
wherein X′ and Y′ are individually selected from CN, CONH₂ and COOR₃′, where R₃′ is selected from H and C1-C12 alkyl, A is selected from Cl, Br, OH and OR₅ wherein R₅ is SO₂R₄ and R₄ is substituted or unsubstituted linear or branched lower alkyl,
with alcoholic ammonia or amine derivative at a temperature in the range of 60-100° C. for 10-24 hours to obtain a compound of Formula III, and
(b) filtration and recrystallization of the compound of Formula III obtained in step (a).

4. The process as claimed in claim 3, wherein the alcoholic ammonia is methanolic ammonia, or ethanolic ammonia; and the amine derivative is selected from alkyl, cycloalkyl, or benzyl amines, carbamates, and sulphonamides.

5. The process as claimed in claim 3, wherein the recrystallization is carried out in a solvent system selected from alcohol as a single solvent, or a two solvent mixtures, comprising a water:alcohol system.

6. The process as claimed in claim 3, wherein the halo-amide of formula V 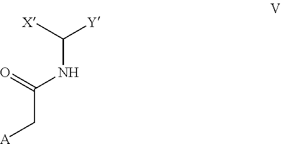
wherein X′ and Y′ are individually selected from CN, CONH₂ and COOR₃′, where R₃′ is selected from H and C1-C12 alkyl, A is selected from Cl, Br, OH and OR₅ wherein R₅ is SO₂R₄ and R₄ is substituted or unsubstituted linear or branched lower alkyl, is prepared by acylation reaction between the compound of formula IV and chloroacetyl chloride of formula VI in presence of base at room temperature, 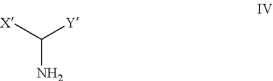
wherein X′ and Y′ are as defined above; 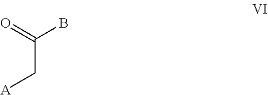
wherein A is as defined above and B is selected from Cl, Br, OH and OR₅ wherein R₅ is SO₂R₄ and R₄ is substituted or unsubstituted linear or branched lower alkyl.

7. A process for preparation of 3,6-dichlorocyano pyrazine of formula II 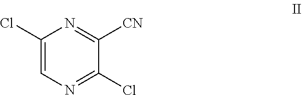
which comprises the steps of:
(a) acylation reaction between the compound of formula IV and chloroacetyl chloride of formula VI in presence of base at room temperature to obtain halo-amide of formula V, 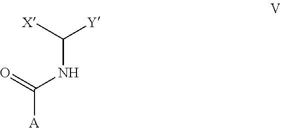
wherein X′ and Y′ are individually selected from CN, CONH₂ and COOR₃′, where R₃′ is selected from H and C1-C12 alkyl, A is selected from Cl, Br, OH and OR₅ wherein R₅ is SO₂R₄ and R₄ is substituted or unsubstituted linear or branched lower alkyl, 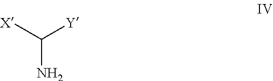
wherein X′ and Y′ are as defined above; 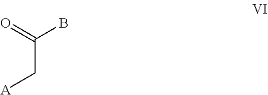
wherein A is as defined above and B is selected from Cl, Br, OH and OR₅ wherein R₅ is SO₂R₄ and R₄ is substituted or unsubstituted linear or branched lower alkyl;
(b) cyclization of halo-amide of formula V with alcoholic ammonia or amine derivative, at a temperature in the range of 60-100° C. for 10-24 hours to obtain a compound of Formula III, 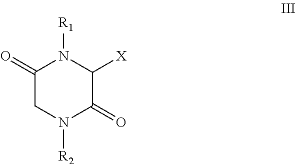
wherein, X is CN, CONH₂ or COOR₂′, R₁, R₂ and R₂′ are individually selected from H, C1-C12 alkyl, COOR₃ and SO₂R₃ wherein R₃ is substituted or unsubstituted linear or branched lower alkyl,
(c) chlorination of 3,6-dioxopiperazine derivative of formula III with POCl₃ and pyridine or PCl₅ at a temperature in the range of 90-140° C. for 4-20 hours, to obtain a compound of Formula II and
(d) purification of the compound of Formula II.

8. The process as claimed in claim 7, wherein the alcoholic ammonia is methanolic ammonia, or ethanolic ammonia; the amine derivative is selected from alkyl, cycloalkyl, or benzyl amines, carbamates, and sulphonamides; and the purification method is selected from crystallization, filtration, and chromatography.

9. A process for preparation of compound of formula I 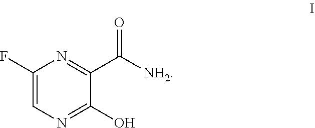 [SMILES: NC(=O)c1nc(F)cnc1O]
which comprises the steps of:
(a) cyclization of halo-amide of formula V 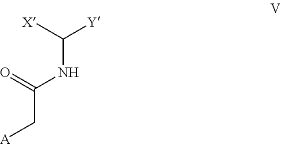
wherein X′ and Y′ are individually selected from CN, CONH₂ and COOR₃′, where R₃′ is selected from H and C1-C12 alkyl, A is selected from Cl, Br, OH and OR₅ wherein R₅ is SO₂R₄ and R₄ is substituted or unsubstituted linear or branched lower alkyl,
with alcoholic ammonia or amine derivatives at a temperature in the range of 60-100° C. for 10-24 hours to obtain a compound of Formula III, 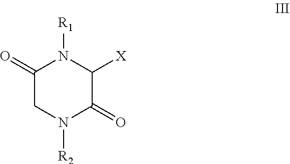
wherein, X is CN, CONH₂ or COOR₂′, R₁, R₂ and R₂′ are individually selected from H, C1-C12 alkyl, COORS and SO₂R₃ wherein R₃ is substituted or unsubstituted linear or branched lower alkyl,
(b) chlorination of 3,6-dioxopiperazine derivative of formula III with POCl₃ and pyridine or PCl₅ at a temperature in the range of 90-140° C. for 4-20 hours, to obtain a compound of Formula II; 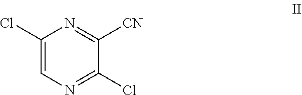
(c) fluorination of compound of formula II obtained in step (b) with potassium fluoride and PTC in a solvent to obtain difluorocyano pyrazine of formula VII at a temperature in the range of 50° C. to 70° C.; 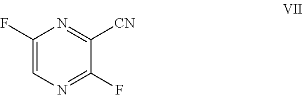
(d) functionalization of aromatic ring in the compound of formula VII obtained in step (c) from fluorine to hydroxy in the presence of sodium acetate to obtain 6-fluoro-3-hydroxypyrazine-2-carbonitrile of formula VIII; 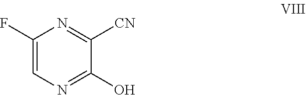
and
(e) hydrolysis of cyano functionality of formula VIII to amide in presence of H₂O₂ and NaOH solution to obtain compound of formula I.

10. The process as claimed in claim 9, wherein the alcoholic ammonia is methanolic ammonia, or ethanolic ammonia; the amine derivative is selected from alkyl, cycloalkyl, or benzyl amines, carbamates, and sulphonamides; and the solvent used in step (c) is selected from DMF, and DMSO.
